(19) United States
(12) Patent Application Publication
(10) Pub. No.: US 2006/0281100 A1
(43) Pub. Date: Dec. 14, 2006

(54) Thiotriphosphate nucleotide dye terminators

(76) Inventors: Gene G.-Y. Shen, Diamond Bar, CA (US); Josephine M. Michael, Placentia, CA (US)

Correspondence Address: BECKMAN COULTER INC.;C/O SHELDON MAK ROSE & ANDERSON, 225 SOUTH LAKE AVENUE, 9TH FLOOR, PASADENA, CA, 91101, US

(21) Appl. No.: 11/153,715

(22) Filed: Jun. 14, 2005

(51) Int. Cl. C12Q 1/68; C12P 19/34; C07H 21/04

(52) U.S. Cl. 435/6; 435/91.2; 536/25.32

(57) ABSTRACT

Fluorescent reporter compounds having a chain terminating (thio)triphosphate nucleotide derivative, a fluorescent dye, and a linker of sufficient length to connect the nucleotide derivative to the fluorescent dye are provided. The fluorescent reporter compounds are used in DNA sequencing reactions and are substantially inactive toward exonuclease digestion.

### BACKGROUND

The present invention relates generally to nucleotide derivatives for use in nucleic acid sequencing, and more particularly, dye labeled chain terminating (thio)triphosphate nucleotides and their method of use in nucleotide (DNA) sequencing.

The chain termination method of nucleic acid sequencing requires the generation of labeled nucleic acid fragments that have a common origin and each terminates with a known base. The labeled nucleic acid fragments are then separated by size (generally by gel electrophoresis) to determine nucleic acid sequence information. Labeled chain terminators can be incorporated into nucleic acid fragments, preferably at the 3′ terminal end, to identify the sequence of a nucleic acid. To be useful as a nucleic acid chain terminator substrate in fluorescence based nucleic acid sequencing, the chain terminator substrate must contain a fluorescent reporter and a nucleotide derivative that is capable of being added to a nucleic acid sequence, but is not capable of being used by a replication enzyme to attach a subsequent nucleotide or nucleotide derivative to the nucleic acid sequence.

Cyanine dyes used to detect biomolecules and in particular as a fluorescent reporter for labeling nucleic acid chain terminator substrates are known. However, these compounds can interfere with the binding or interaction of the nucleotide derivative with the replication enzyme, are unstable, are difficult to synthetically manufacture, or have a fluorescent detection wavelength that is problematic for automated systems. In addition, DNA sequencing reactions using labeled 3′ terminators can have false stop products that interfere with the analysis of the DNA reaction products.

Therefore, there is a need for fluorescently labeled nucleic acid chain terminators that do not interfere with. nucleotide replication, that are stable, and have a fluorescent detection wavelength that is amenable to automated systems. There is also a need for fluorescently labeled nucleic acid chain terminators that are stable toward methods of eliminating the false stop products of a sequencing reaction.

Further information on known fluorescent probes used to detect biomolecules, and cyanine dyes in general can be found in Flanagan, J. H., et al., Bioconjugate Chem. 8:751-756 (1997); Ludwig, J. et al., J. Org. Chem., 54:631-635 (1989); Mujumdar, R. B., et al., Bioconjugate Chem., 4:2 105-111 (1993); Mujumdar, R. B., et al., Cytometry, 10:11-19 (1989); Mujumdar, S. R., et al., Bioconjugate Chem., 7:356-362 (1996); Ozmen, B., et al., Tetrahedron Letters, 41:9185-9188 (2000); Shealy, D. B., et al., Anal. Chem. 67:247-251 (1995); Southwick, P. L., et al., Cytometry, 11418-430 (1990); Strekowski, L., et al., J. Org. Chem., 57:4578-4580 (1992); and Williams, R. J., et al., Anal. Chem., 65:601-605 (1993); and U.S. Pat. Nos. 5,453,505; 5,571,388; and 6,002,003.

### SUMMARY

The present invention if for fluorescent reporter compounds having a (thio)triphosphate nucleotide derivative are provided. The compounds contain a (thio)triphosphate nucleotide derivative, a fluorescent dye, and a linker of sufficient length to connect the nucleotide derivative to the fluorescent dye. The fluorescent reporter compounds are used in DNA sequencing reactions and are substantially inactive toward exonuclease digestion.

According to the present invention, the invention provides compounds of the formula:

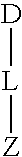

where Z is a (thio)triphosphate nucleotide derivative; D is a fluorescent dye; and L is a linker of sufficient length to connect the (thio)triphosphate nucleotide to the fluorescent dye, such that the fluorescent dye does not significantly interfere with the overall binding and recognition of the nucleotide derivative by a nucleic acid replication enzyme.

Preferably, D is a fluorescent cyanine dye that has an emission wavelength in the near infrared. More preferably, the D is a fluorescent cyanine dye of the formula:

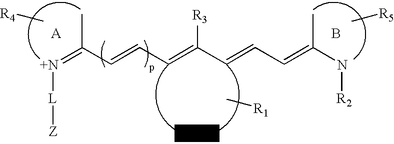

where

A and B are each independently ring structures having sufficient atoms to form a cyanine nuclei;

n is 0, 2, 3, or 4;

p is 0 or 1;

R₁ is selected from the group consisting of C₁-C₄ alkyl, C₁-C₄ haloalkyl, halogen, and hydroxy with the proviso that R₁ is not present when n is 0;

R₂ is selected from the group consisting of C₁-C₂₀ alkyl and hydrogen, wherein the C₁-C₂₀ alkyl is unsubstituted or substituted with C₁-C₄ alkoxy, C₁-C₄ alkoxycarbonyl, C₁-C₄ alkyl, amine, amide, C₁-C₄ carboxy, C₁-C₄ carboxylate, halogen, hydroxy, and sulfonate;

R₃ is selected from the group consisting of hydrogen, halogen, OR₇, SR₇, NR₇R₈, C₁-C₆ alkyl, C₁-C₆ alkylene, C₃-C₆ cycloalkyl, C₃-C₆ cycloheteroalkyl, C₃-C₆ cycloalkylene, C₃-C₆ cycloheteroalkylene, phenyl, biaryl, heteroaryl, or heterobiaryl, wherein the C₁-C₆ alkyl, C₁-C₆ alkylene, C₃-C₆ cycloalkyl, C₃-C₆ cycloheteroalkyl, C₃-C₆ cycloalkylene, C₃-C₆ cycloheteroalkylene, phenyl, biaryl, heteroaryl and heterobiaryl groups are unsubstituted or substituted with C₁-C₄ alkyl, C₁-C₄ haloalkyl, halogen or hydroxy;

R₄ and R₅ are each independently selected from the group consisting of C₁-C₄ alkoxy, C₁-C₄ alkoxycarbonyl, C₁-C₄ alkyl, amine, amide, C₁-C₄ carboxy, C₁-C₄ carboxylate, halogen, hydroxy, and sulfonate;

R₇ and R₈ are each independently selected from the group consisting of hydrogen, C₁-C₆ alkyl, C₃-C₆ cycloalkyl, phenyl, biaryl, heteroaryl, and heterobiaryl, wherein the C₁-C₆ alkyl, C₁-C₆ cycloalkyl, phenyl, biaryl, heteroaryl, and heterobiaryl groups may be substituted with halogen, OH, C₁-C₄ alkyl, and C₁-C₄ haloalkyl, or when R₃ represents NR₇R₈, R₇ and R₈ may be taken together to form an optionally substituted C₃-C₆ aliphatic or C₃-C₆ aromatic heterocyclic ring.

In the compounds represented by the formula D-L-Z, the fluorescent reporter compounds are preferably a-(thio)triphosphate dideoxynucleotides, where more preferably, Z is represented by one of the following a-(thio)triphosphate dideoxynucleotides:

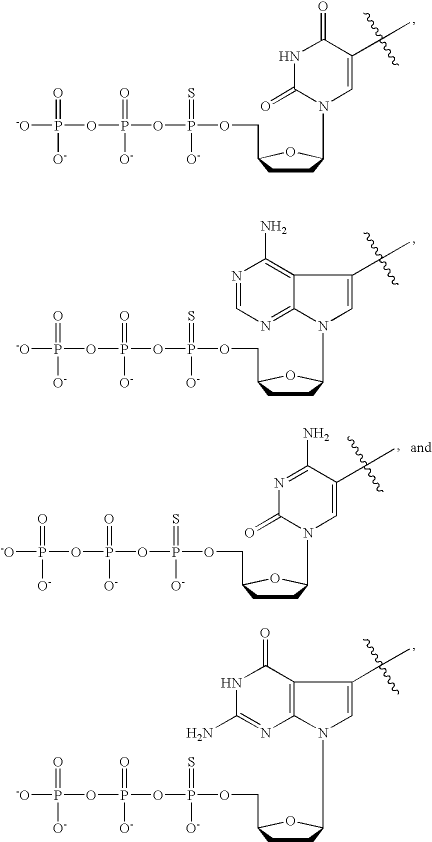

Also in the compounds represented by the formula D-L-Z, L is preferably a linker of the formula:

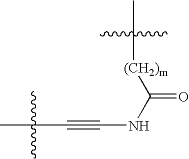

where m is an integer from 1 to 20.

Preferred compounds of the formula D-L-Z are represented by the following formulas:

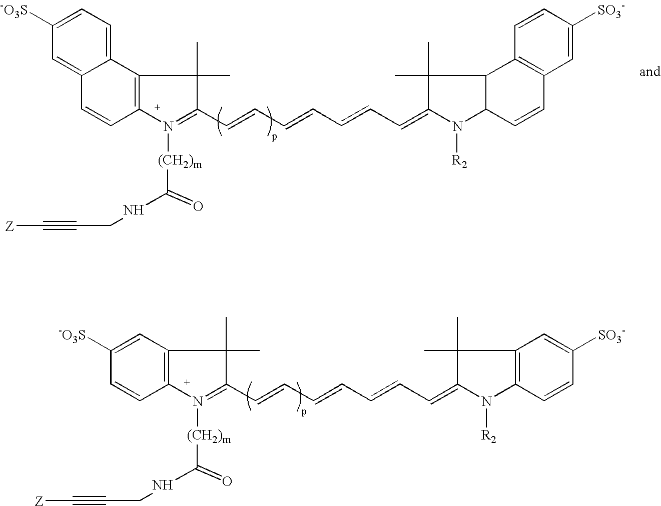

where Z is defined as described herein; m is an integer from 1 to 20; p is 0 or 1; and

R₂ is selected from the group consisting of C₁-C₂₀ alkyl and hydrogen, wherein the C₁-C₂₀ alkyl is unsubstituted or substituted with C₁-C₄ alkoxy, C₁-C₄ alkoxycarbonyl, C₁-C₄ alkyl, amine, amide, C₁-C₄ carboxy, C₁-C₄ carboxylate, halogen, hydroxy, and sulfonate.

The compounds of the present invention are also represented by the following formula:

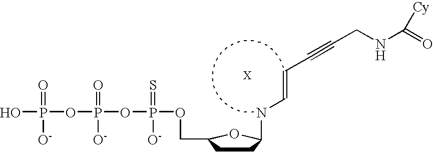

where X are the atoms sufficient to make up a heterocyclic base; and Cy is a fluorescent dye. Preferably, Cy is a cyanine dye.

According to another embodiment, the invention is a method of nucleic acid sequence analysis. According to this embodiment, a fluorescent reporter labeled compound, as described herein, is reacted with a first nucleic acid sequence to produce a second nucleic acid sequence labeled with the fluorescent reporter-labeled compound. Then, the reporter on the second nucleic acid sequence is detected.

According to yet another embodiment, the invention is a method for determining the base sequence of a target DNA. According to this embodiment, a mixture of fluorescent reporter labeled compounds, such as those as described herein, is provided, where the mixture of fluorescent reporter labeled compounds corresponding to each of the four DNA bases. Then, a DNA template corresponding to the target DNA is provided. The DNA template is reacted with a DNA primer and the DNA template and DNA primer are extended with a replication enzyme, a mixture of DNA nucleotides, and the mixture of fluorescent reporter labeled compounds. This produces a plurality of DNA fragments, each DNA fragment having a fluorescent reporter labeled compound attached to the 3′-terminal residue of the DNA fragment. Next, the plurality of fluorescent reporter labeled DNA fragments are separated and the fluorescent reporter on each separated fluorescent reporter labeled DNA fragment is detected to determine the base sequence of the target DNA.

Preferably, at least one of the fluorescent reporter labeled (thio)triphosphate nucleotide compounds is labeled with a cyanine dye and/or at least one of the compounds is labeled with a dye having a near infrared fluorescence. More preferably, four different fluorescent reporter (thio)triphosphate nucleotide compounds are provided, each fluorescent reporter labeled (thio)triphosphate nucleotide compound being labeled with a dye having a near infrared fluorescence. Most preferably, each of the four different fluorescent reporter labeled (thio)triphosphate nucleotide compounds is labeled with a cyanine dye.

### DESCRIPTION

According to one embodiment of the present invention, there is provided fluorescent reporter compounds having a chain terminating (thio)triphosphate nucleotide derivative, a fluorescent dye, and a linker of sufficient length to connect the nucleotide derivative to the fluorescent dye, such that the fluorescent dye does not significantly interfere with the overall binding and recognition of the nucleotide derivative by a nucleic acid replication enzyme.

The compounds of the present invention incorporate a sulfur moiety into the phosphate portion of a nucleotide, i.e., thio-derivatized nucleotides. This feature renders the fluorescent reporter compounds inactive toward exonuclease digestion. Thus, when the fluorescent reporter compounds of the present invention are incorporated into a DNA sequencing reaction, false stop products can be eliminated using an exonuclease. This leaves the DNA fragments that have incorporated the thio-derivatized nucleotides as clean reaction products that can then be analyzed for DNA sequencing information.

Further, the fluorescent reporter compounds do not significantly interfere with nucleotide replication, are stable when stored over time, and have fluorescent detection wavelengths in the near infrared region, which is amenable to existing automated systems.

As used in this disclosure, the terms listed below have the following meanings.

The term “cyanine nuclei” means the carbon, hydrogen, and hetero- atoms necessary to complete the conjugated system that makes up a fluorescent cyanine chromophore. Cyanine nuclei that can be used in the fluorescent-labels according to the present invention are known to those skilled in the art. Examples of cyanine nuclei include substituted or unsubstituted thiazole, benzothiazole, napthothiazole, benzoxazole, napthoxazole, benzolselanazole, napthoselenazole, indole, and benzoindole rings.

The term “heterocyclic base” means a purine or pyrimidine base capable of acting as a recognition element by a replication enzyme used in a nucleic acid synthesis.

The term “nucleotide derivative” means a compound having a heterocyclic-base, a sugar, and a phosphate functionality that is capable of being added to a nucleic acid sequence, but is not capable of being used by a replication enzyme to attach a subsequent nucleotide or nucleotide derivative to the nucleic acid sequence.

The term “nucleoside derivative” means a nucleotide derivative minus the phosphate functionality.

The term “phosphate functionality” means a mono-, di-, or tri-phosphate, or a phosphate analog such as an alpha-(thio)triphosphate, that when joined to a nucleoside derivative forms a nucleotide derivative that is capable of being used by a replication enzyme to attach the nucleotide derivative to a nucleic acid sequence.

The term “sugar” means a 5- or 6-membered heterocycle that when incorporated into a nucleic acid sequence is not capable of being used by a replication enzyme to attach a subsequent nucleotide or nucleotide derivative to the nucleic acid sequence.

The term “comprise” and variations of the term, such as “comprising” and “comprises,” are not intended to exclude other additives, components, integers or steps.

In one embodiment, the present invention is a fluorescent reporter compound according to Formula (I) below:

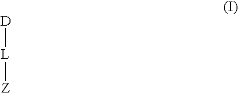

In the above Formula (I), “Z” represents a chain terminating (thio)phosphate nucleotide derivative having a heterocyclic-base, a sugar, and a phosphate functionality;

L is a linker of sufficient length to connect the (thio)triphosphate nucleotide to the fluorescent dye, such that the fluorescent dye does not significantly interfere with the overall binding and recognition of the nucleotide derivative by a nucleic acid replication enzyme; and

D is a fluorescent dye.

The nucleotide derivatives, represented as “Z” in the above Formula (I), are generally comprised of a heterocyclic-base, a sugar, and a (thio)phosphate functionality.

The heterocyclic-base is the portion of the nucleotide derivative that functions as the recognition element in nucleotide synthesis. Generally, these are a purine or pyrimidine base that corresponds to a natural nucleic base. Examples of heterocyclic-bases including cytocine, deazaadenine, deazaguanine deazahypoxanthine, and uracil are shown below.

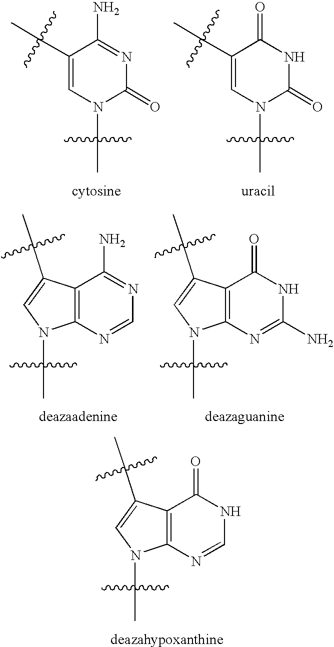

Other heterocyclic-bases that can act as the recognition element in nucleic acids such as 8-aza-7-deazapurines and 3,7-dideazaadenine can also be used.

The “sugar” portion of the nucleotide derivative corresponds to the deoxyribofuranose structural portion in the natural enzyme substrate. The sugar portion of the nucleotide derivative used in the fluorescent-labeled reporter compounds is generally a modified 5- or 6-membered heterocycle such as a furanose that is not capable of being used by a replication enzyme to attach a subsequent nucleotide or nucleotide derivative to the nucleic acid sequence.

The “phosphate functionality” part of the nucleotide derivative used in the fluorescent labeled reporter compound according to the present invention, is a mono-, di-, or tri-(thio)phosphate functionality and can have varying structures as will be understood by those of skill in the art with reference to this disclosure. However, according to a preferred, but not required aspect of the present invention, the (thio)triphosphate is substituted in the alpha-(α-) position. More preferably, Z is an α-(thio)triphosphate dideoxynucleotide, and most preferably, Z is one of the following α-(thio)triphosphate dideoxynucleotides:

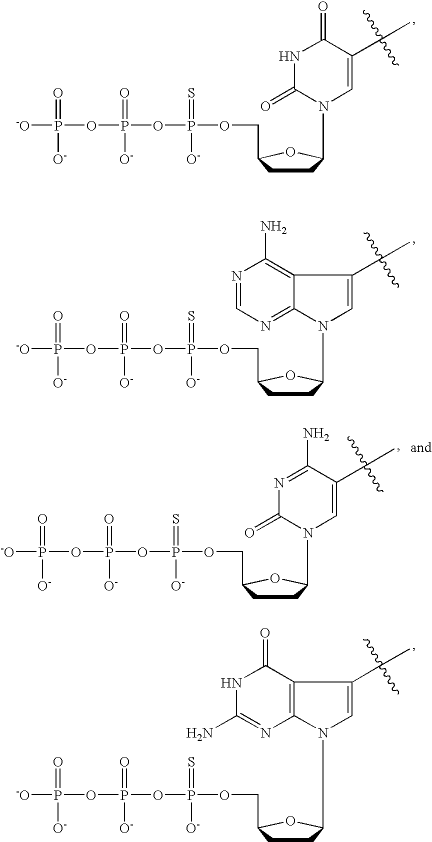

The fluorescent reporter compounds of the present invention also have a linker, shown as “L” in Formula (I) above, which represents a linker of sufficient length to connect the nucleotide derivative and the fluorescent dye such that the fluorescent dye and linker do not significantly interfere with the overall binding or recognition of the nucleotide derivative by a nucleic acid replication enzyme. Such linker groups are known to those of skill in the art and can be selected for use in the compounds according to the present invention, as will be understood by those of skill in the art with reference to this disclosure. In a preferred, but not required embodiment, L is a linker of the formula:

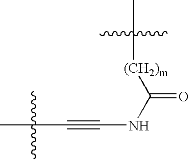

wherein m is an integer from 1 to 20.

The fluorescent reporter compounds of the present invention also have a fluorescent dye, shown as “D” in Formula (I) above. Suitable fluorescent dyes that can be used in the fluorescent reporter compounds of the present invention are known to those of skill in the art. Preferably, the fluorescent dyes have absorbance and emission spectra in the near infrared (NIR) region, i.e., absorbance and emission just beyond the visible region (e.g., from about 600 nm to about 1600 nm). Examples of suitable fluorescent dyes include cyanine dyes (e.g., phthalocyanines and carbocyanines) and squaraines, etc. See, e.g., Masaru Matsuoka, Infrared Absorbing Dyes, Plenum press, New York (1990).

More preferably, the fluorescent reporter compounds are cyanine dyes according to Formula (II): 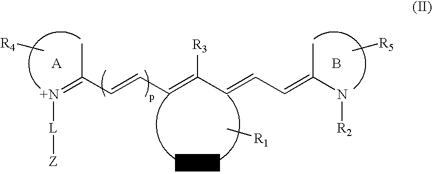
wherein

A and B are each independently ring structures having sufficient atoms to form a cyanine nuclei, preferably, A and B are indole or benzoindole rings;

n is 0, 2, 3, or 4;

p is 0 or 1;

R₁ is selected from the group consisting of C₁-C₄ alkyl, C₁-C₄ haloalkyl, halogen, and hydroxy with the proviso that R₁ is not present when n is 0;

R₂ is selected from the group consisting of C₁-C₂₀ alkyl and hydrogen, wherein the C₁-C₂₀ alkyl is unsubstituted or substituted with C₁-C₄ alkoxy, C₁-C₄ alkoxycarbonyl, C₁-C₄ alkyl, amine, amide, C₁-C₄ carboxy, C₁-C₄ carboxylate, hydroxy, and sulfonate;

R₃ is selected from the group consisting of hydrogen, halogen, OR₇, SR₇, NR₇R₈, C₁-C₆ alkyl, C₁-C₆ alkylene, C₃-C₆ cycloalkyl, C₃-C₆ cycloheteroalkyl, C₃-C₆ cycloalkylene, C₃-C₆ cycloheteroalkylene, phenyl, biaryl, heteroaryl, or heterobiaryl, wherein the C₁-C₆ alkyl, C₁-C₆ alkylene, C₃-C₆ cycloalkyl, C₃-C₆ cycloheteroalkyl, C₃-C₆ cycloalkylene, C₃-C₆ cycloheteroalkylene, phenyl, biaryl, heteroaryl and heterobiaryl groups are unsubstituted or substituted with C₁-C₄ alkyl, C₁-C₄ haloalkyl, halogen, and hydroxy;

R₄ and R₅ are each independently selected from the group consisting of C₁-C₄ alkoxy, C₁-C₄ alkoxycarbonyl, C₁-C₄ alkyl, amine, amide, C₁-C₄ carboxy, C₁-C₄ carboxylate, halogen, hydroxy, and sulfonate;

R₇ and R₈ are each independently selected from the group consisting of hydrogen, C₁-C₆ alkyl, C₃-C₆ cycloalkyl, phenyl, biaryl, heteroaryl, and heterobiaryl, wherein the C₁-C₆ alkyl, C₁-C₆ cycloalkyl, phenyl, biaryl, heteroaryl, and heterobiaryl groups may be substituted with halogen, hydroxy, C₁-C₄ alkyl, and C₁-C₄ haloalkyl, or when R₃ represents NR₇R₈, R₇ and R₈ may be taken together to form an optionally substituted C₃-C₆ aliphatic or C₃-C₆ aromatic heterocyclic ring.

In a preferred, but not required embodiment, D and L, taken together form a compound according to one of Formulas (III)-(VI) below:

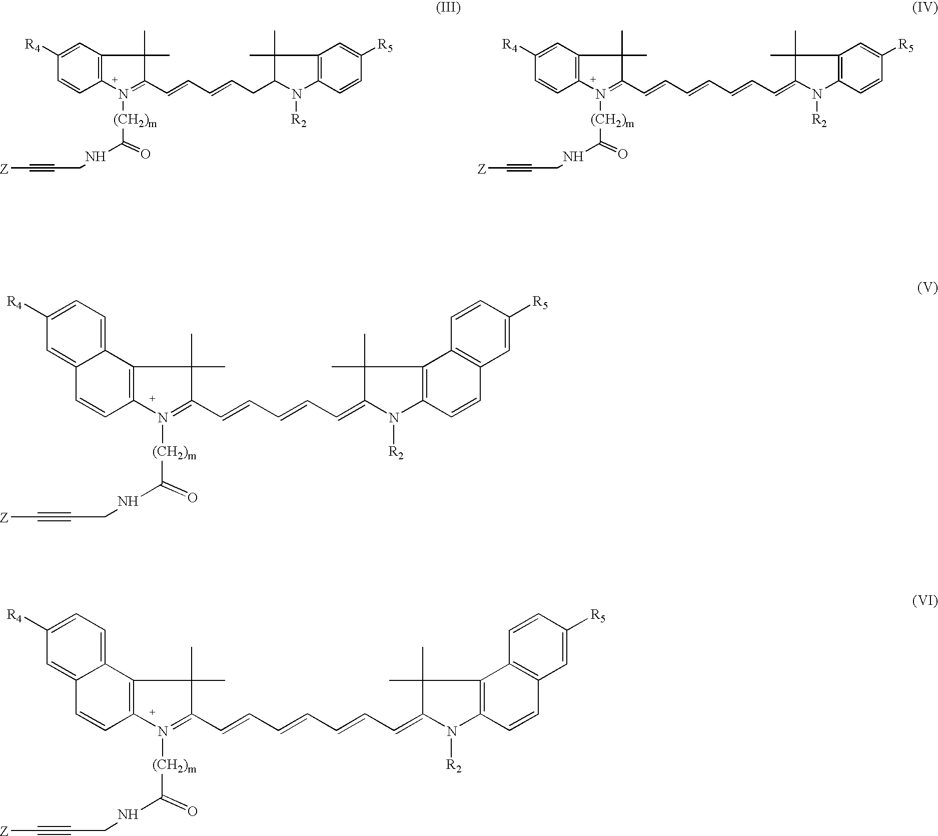

where Z, R₂, R₄, R₅, and m are defined as above.

Preferred fluorescent reporter compound are shown below as Compounds 1-4.

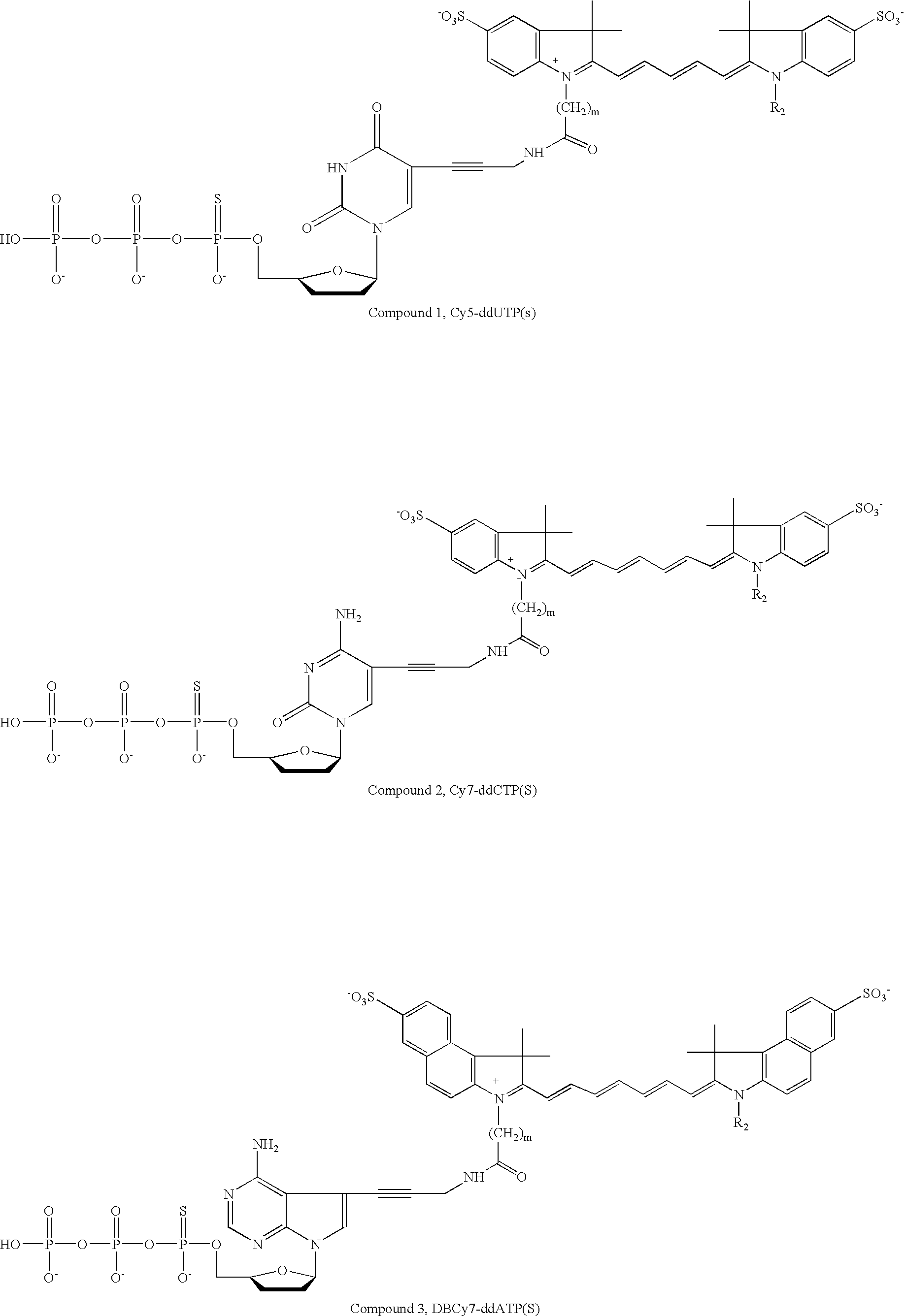

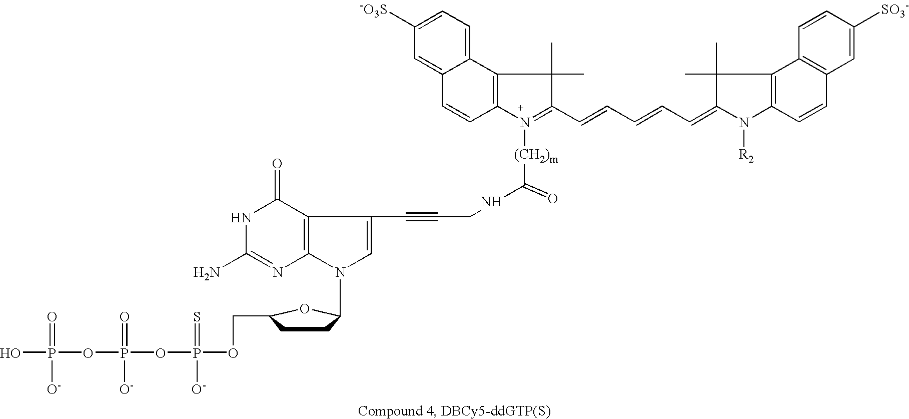

Fluorescent reporter compounds can be prepared by thio conversion of known dye terminators. According to the present invention, existing dye terminators, such as those disclosed in U.S. Pat. No. 6,002,003, which is incorporated herein by reference, can be used as a starting material and then converted to the α-thio derivative by treatment with alkaline phosphatase. An example of the conversion of the known dye terminator, Compound 5, Cy5-ddUTP, to the α-thio derivative, Compound 1, Cy5ddUTP(s) is shown below in Scheme I.

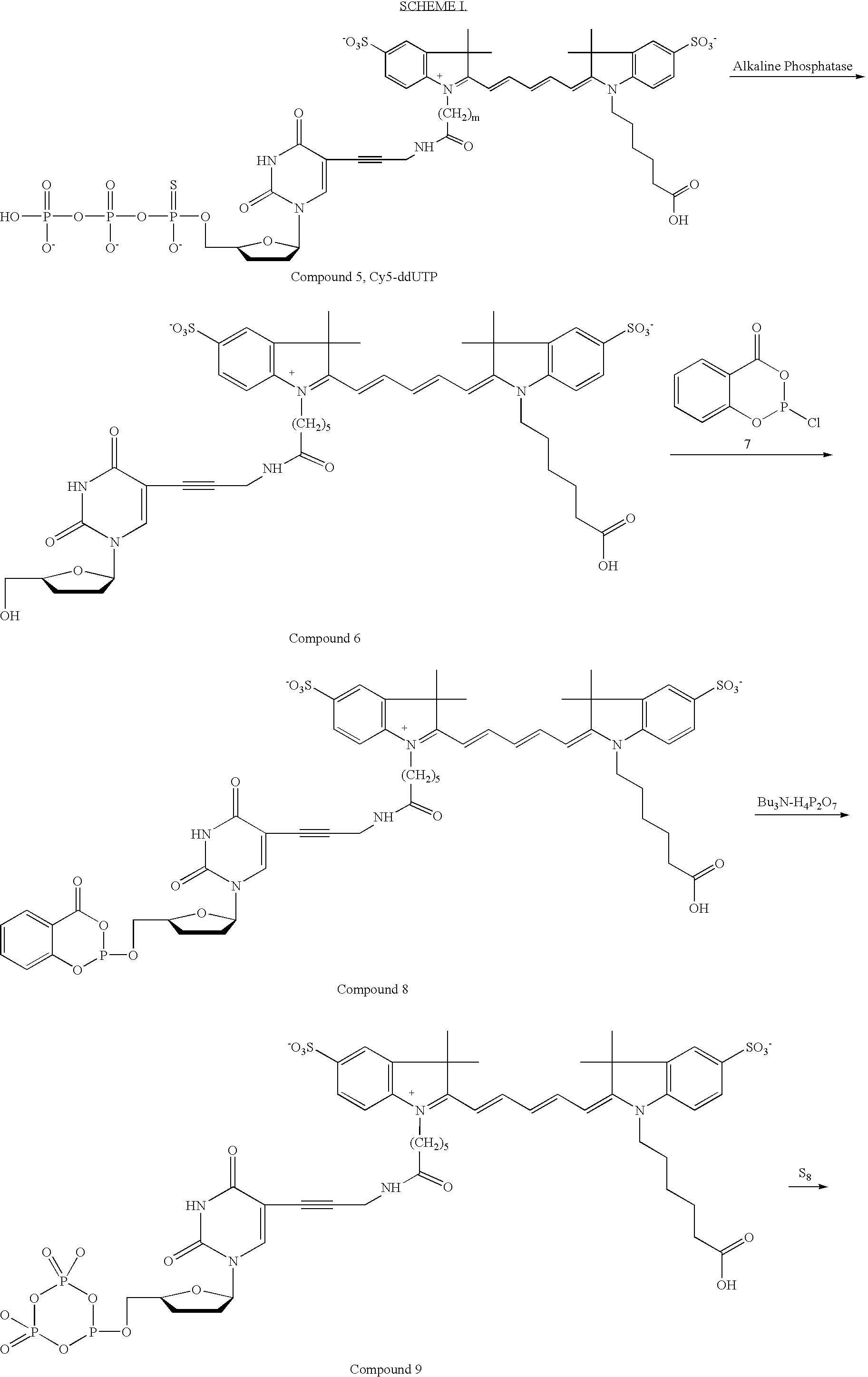

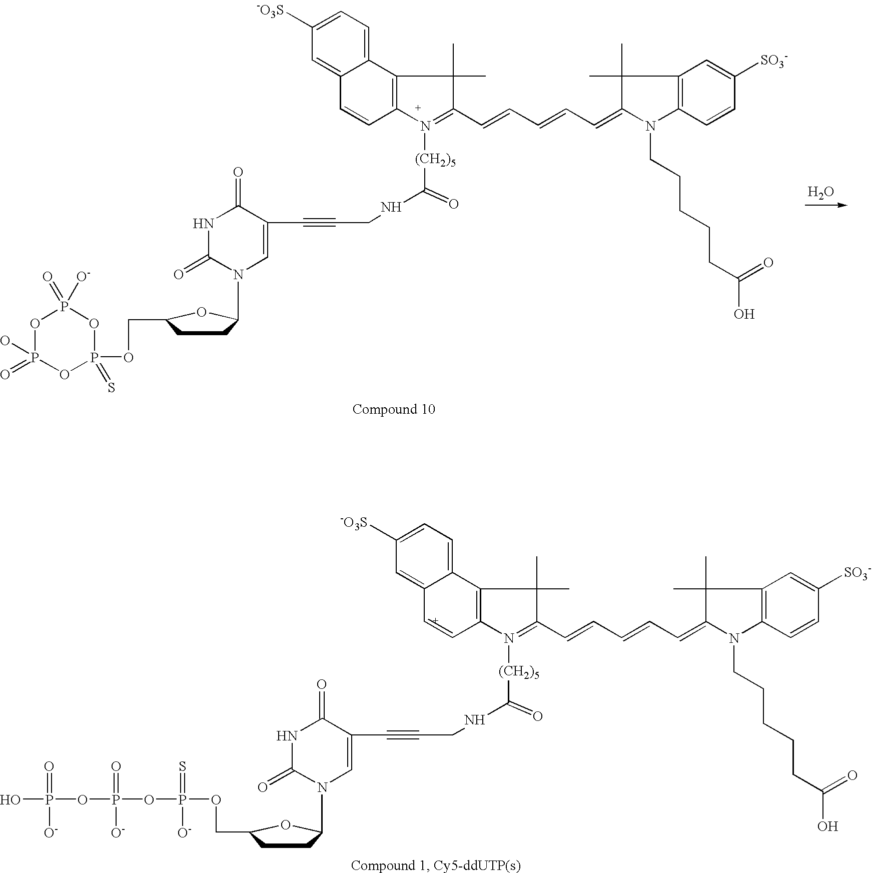

As shown in Scheme I, the dye-terminator Cy5-ddUTP (Compound 5) was treated with alkaline phosphatase to digest off the triphosphate to the non-phosphate Cy5-ddU (Compound 6) through the di-phosphate and mono-phosphate intermediates. The progress of the enzymatic reaction was monitored and followed by CE/LIF to completion and the crude reaction mixture was then added to a size exclusion column (G-25) and eluted with aqueous media to remove the enzyme and to collect the blue band of product, Cy5-ddU (Compound 6).

Compound 6 was then treated with 2-chloro-4H-1,3,2-benzodioxaphosphorin-4-one (7) according to the literature reference to give the intermediate 8. (For reference, see, Ludwig, J. Eckstein, F. J. Org. Chem., 1989, 54, 631-635). The following steps including a) treatment of 8 with pyrophosphate to the cyclic nucleoside 9; b) oxidation of 9 with sulfur to intermediate nucleoside 5′-(l-thiocyclotriphosphate) 10; and c) the nucleophilic attack of water to the intermediate 10 to give the desired product 1, were carried out in situ in the same flask without purification of either intermediate. The crude reaction product from above was subjected to capillary electrophoresis analysis and compared with that of the starting material. Several new peaks together with the starting material were observed, demonstrating the formation of the desired product.

In an alternate method of preparation, fluorescent reporter compounds can be prepared by a direct synthetic approach. According to this embodiment, fluorescent reporter compounds, can be prepared by coupling activated BOSu (N-hydroxyphthalimide) ester cyanine dyes with base modified dideoxy nucleotides as shown in Scheme II.

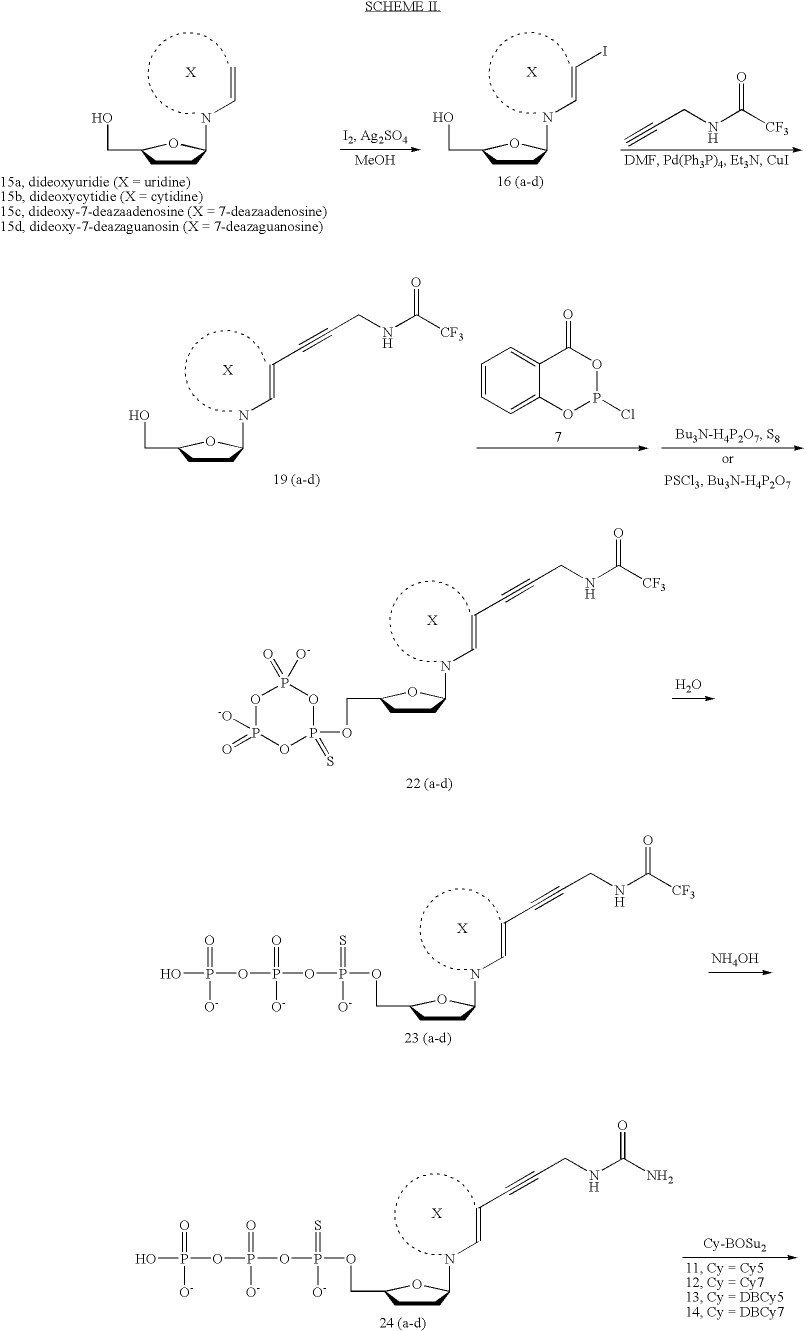

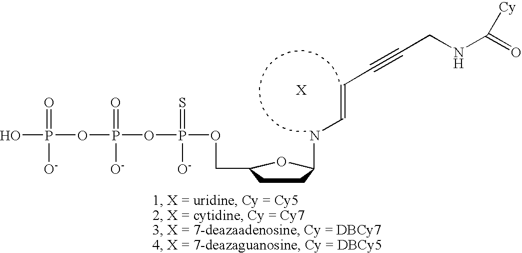

According to this embodiment, fluorescent reporter compounds can be prepared by first converting a starting dideoxynucleoside (Compounds 15a -15d) to the corresponding iodo-dideoxynucleoside (Compounds 16a-16d) by treatment with iodine and silversulfate. A propargyl amino nucleoside derivative (Compounds 19a-d) is then prepared by coupling the iodo-dideoxynucleoside to a TFA protected propargyl amino side arm using palladium coupling chemistry.

Intermediate nucleoside 5′-(1-thiocyclotriphosphate) compounds (Compounds 22a and 22c) can be prepared by reaction of a propargyl amino nucleoside compound (Compounds 19a and 19c) with 2-chloro-4H-1,3,2-benzodioxaphosphorin-4-one (7) to produce nucleoside Compounds 20a and 20c (not shown). Treatment of these nucleosides with pyrophosphate gives the corresponding cyclic nucleoside compound (Compounds 21a and 21c (not shown)). Oxidation of the cyclic nucleoside with sulfur produces nucleoside 5′-(1-thiocyclotriphosphate) compounds (Compounds 22a and 22c).

In an alternate preparation, intermediate nucleoside 5′-(-tlhiocyclotriphosphate) compounds (Compounds 22b and 22d) can be prepared by reaction of the propargyl amino nucleoside compounds 19b and 19d with PSCl₃ to produce a thio-phosphochloride nucleoside compound (Compounds 21b and 21d (not shown). Subsequent reaction of the thio-phosphochloride nucleoside compounds with pyrophosphate produces nucleoside 5′-(1-thiocyclotriphosphate) compounds (Compounds 22b and 22d).

Nucleophilic attack of water on Compounds 22a-22d produces an intermediate alpha-nucleotide compound (Compounds 23a-23d). The alpha-(thio)triphosphate compound is then deprotected with ammonium hydroxide to give the amino alpha-(thio)triphosphate nucleotide compound (Compounds 24a-24d).

Fluorescent reporter compounds (Compounds 1-4) can then be prepared by reacting an amino [symbol] -(thio)triphosphate nucleotide compound (Compounds 24a-24d) with excess of an activated BOSu cyanine dye ester (Compounds 11-14, shown below).

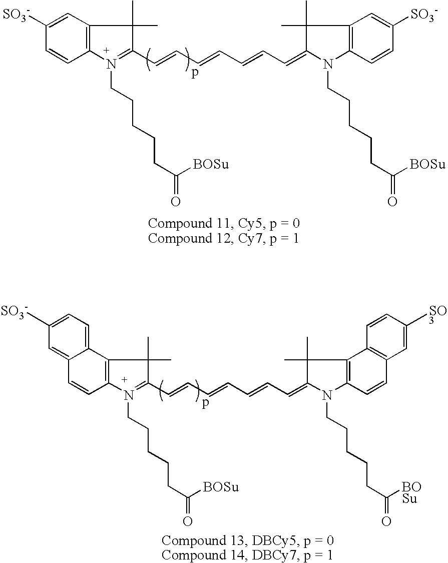

The syntheses of cyanine dyes and corresponding dye esters, such as those shown above, is described in U.S. Pat. No. 6,002,003.

In another embodiment, the present invention is a method of nucleic acid sequence analysis. In one embodiment, the method comprises reacting a fluorescent reporter labeled compound as described herein with a first nucleic acid sequence to produce a second nucleic acid sequence labeled with the fluorescent reporter-labeled compound. Then, the reporter on the second nucleic acid sequence is detected.

In another embodiment, the present invention is a method for determining the base sequence of a target DNA. According to this embodiment, a mixture of fluorescent reporter labeled compounds, such as those described herein, is provided. The mixture of fluorescent reporter labeled compounds corresponds to each of the four DNA bases. Preferably, the fluorescent dyes contained in the fluorescent reported labeled compounds have spectroscopically distinguishable emission spectra (preferably with absorbance maxima separated by about 30 nm to 35 nm) in the near infrared region (i.e., from about 600 nm to about 1600 nm, and more preferably from about 600 nm to about 900 nm). Most preferably, the four fluorescent dyes have excitation wavelengths that correspond to commercially available excitation lasers. Also preferably, the fluorescent dyes are used in a combination of four fluorescent dyes that have spectroscopically distinguishable emission spectra that are excited by two different excitation wavelengths, each in the near infrared region. The absorbance maxima of the fluorescent dyes are grouped into two pairs, the two pairs of dyes being separated by about 100 nm, such that two diode laser sources can be used to excite the two pairs of dyes. Preferred laser sources have excitation wavelengths of about 650 nm and about 780 nm.

Next, a DNA template, corresponding to the target DNA is combined in the reaction mixture. Then, the DNA template is reacted with a DNA primer. The DNA template and DNA primer are extended with a replication enzyme, a mixture of DNA nucleotides, and the mixture of fluorescent reporter labeled compounds. This reaction produces DNA fragments having a fluorescent reporter labeled compound attached to the 3′-terminal residue of the DNA fragment. The fluorescent reporter labeled DNA fragments are then separated. Preferably, the labeled DNA fragments are separated by electrophoresis, more preferably by capillary gel electrophoresis. After separation, the fluorescent reporter on each separated fluorescent reporter labeled DNA fragment is detected. This information is then analyzed to determine the base sequence of the target DNA.

Methods of detecting and analyzing fluorescent reporters for determining DNA sequences are known to those of skill in the art. A preferred detection and analysis system is an automated sequencer such as the Beckman CEQ™ autonmated sequencer. However, other detection and analysis systems can be used according to the present invention as will be understood by those of skill in the art with reference to this disclosure.

### EXAMPLES

General Procedures

Chromatography.

High pressure liquid chromatography (HPLC) was performed on a Beckman model 100A solvent delivery system equipped with an Altex injector and a Waters 990 photodiode array detector. A Beckman ultrasphere ODS (5 mm particle size, 4.6 mm (inner diameter)×4.5 cm (length) column was used for reversed phase conditions unless otherwise noted. All HPLC grade solvents were used without further purification. Thin-layer chromatography (TLC) was performed using aluminum sheets coated with silica gel (EM silica gel 60 F₂₅₄). Compounds were detected by their color bands or by UV light.

Ultraviolet Spectra.

Unless otherwise stated, ultraviolet spectra (UV) were recorded for samples dissolved in MeOH or water contained in a one cm path length quartz cell using a Beckman DU-7400 spectrophotometer.

Capillary Electrophoresis (CE).

The purity the dye terminators prepared, as described below, was assessed by capillary electrophoresis using a 27 cm in length capillary column having a 25 mm (inner diameter), unless otherwise stated. The running buffer was 100 mM sodium borate pH 10.2. The CE was run at 20 KVs. The laser induced fluorescence (CE/LIF) was measured on a P/ACE™ 5500 using a 633 nm diode laser and a 633 nm notch filter. A 665 nm emission filter was used for Cy5-ddUTP(S) and DBCy5-ddGTP(S). A 670 nm diode laser and 780 emission filter was used for Cy7-ddCTP(S) and an 810 emission filter was used for DBCy7-ddATP(S).

Reaction and Workup Procedure.

All operations were conducted under either an inert atmosphere of commercial purified nitrogen or an anhydrous condition with a Drierite tube. The anhydrous solvents were purchased from Aldrich and were used without further purification. Reaction products were normally concentrated by rotary evaporation of volatile solvents at reduced pressure using a water aspirator.

### Example 1

### Preparation of Compound 1 via Enzymatic Digestion

Referring again to Scheme I, Cy5-ddUTP (Compound 5) (0.75 mL, 171 nmol, 228 μM) was added 5 μL of enzyme (350 u/mL) in a vial. Digestion was continued for 2 hours at room temperature. CE analysis indicated the digestion was complete after 2 hours and only the non-phosphate Cy5-ddU was observed. The produce was purified and separated from the enzyme using size exclusion chromatography by loading the crude product on top of a 10 mL G-25 column and eluting with water. The blue band was collected and concentrated to dryness.

Pyridine (˜1 mL) was then added to the crude Cy5-ddU prepared above from the enzymatic digestion of Cy5-ddUTP and concentrated to dryness under high vacuum. The process was repeated ensure the dryness of the material Pyridine (2 μL) was then added and the flask was flushed with nitrogen. Dioxane (6 μL) was added to the flask followed by 2 μL of a freshly prepared 1 M solution of 2-chloro-4H-1,2,3-dioxaphosphorin-4-one (Compound 7) in anhydrous dioxane. After 30 min, 2 μL of 0.5 M solution of bis(tri-n-butylammonium)pyrophosphate in a 3:1 ratio of DMF and tri-n-butylamine was added at room temperature and let stand for 25 min with occasional vertex. A suspension of S₈ (˜1 mg) in DMF (0.5 μL) was then added to the reaction mixture and let stand at room temperature for 30 min with occasional vertex. TEAB buffer (100 μL of 1 M TEAB) was added then added to the reaction flask and the reaction mixture stood overnight. The solvent was then evaporated to dryness under high vacuum. The residual blue trace of product was re-dissolved in small amount of 1:1 MeOH/water. CE/LIF analysis of the crude product showed product peak together with the starting material. No attempt was made to isolate the product from the crude mixture.

### Example 2

### Synthetic Preparation of Compound 1

Preparation of ddUTP(S)-propargylamine (Compound 24a, ddUTP(S)-PA—NH₂). Referring again to Scheme II, Compound 15a, iodo-dideoxyuridine, obtained by known procedures, Pfitzer et al, J. Org. Chem., 29:1508 (1864); Robins et al., Can. J. Chem., 60:554 (1982) was reacted with a propargyl amino compound using palladium coupling chemistry to produce Compound 19a in 85% yield.

Compound 19a (111.0 mg, 300 μmol) and pyridine (10 mL) were then added to an oven dried 25 mL round bottom flask. The reaction product was concentrated to dryness under high vacuum and another 10 mL of pyridine was then added to the reaction flask, concentrated to dryness, and then dried under high vacuum pump for 2 hours. Pyridine (300 μL) was then added to the flask and the flask was briefly flushed with nitrogen via a needle through a rubber septum in the flask. Dioxane (900 μL) was then added to the flask, followed by 330 μL of a freshly prepared 1 M solution of 2-chloro-4H-1,2,3-dioxaphosphorin-4-one (Compound 7) in anhydrous dioxane. After 30 min, 1200 μL of a 0.5 M solution of bis(tri-n-butylammonium)pyrophosphate in 3:1 ratio of DMF and tri-n-butylamine was added and the flask was stirred at room temperature for 25 minutes under nitrogen. A suspension of S₈ (20 mg) in DMF (600 μL) was then added and the reaction was stirred for 30 min. TEAB buffer (1,000 μL of 1 M TEAB) was then added and the reaction was stirred overnight. The solvent was then evaporated from the reaction under high vacuum to dryness.

The crude reaction product was purified on a DEAE column (A-25, 0.05 M TEAB, 2.5 cm×13 cm) using a gradient TEAB buffer (0.05 M to 1 M) (1200 mL). Each fraction (8 mL) of the solution was collected and checked with CE. The fractions containing product (collected after eluting with 0.66 M to 0.75 M of TEAB buffer) were combined and concentrated to dryness. The crude product was then dissolved in water (6 mL) and to it was added NH₄OH (6 mL) to de-protect the TFA group from the amine. The reaction was then stirred overnight and the solvent was evaporated to dryness. The product was re-dissolved in water and analyzed with CE (15 KV, 100 mM borate buffer, pH 10.2). The solution was treated with 1.5 mL of 1 M Na₂CO₃ and evaporated to dryness to remove residual NH₄OH. The product was then re-dissolved in water (2 mL) and the UV absorbance (1/200 dilution) was measured. The UV absorbance at λmax 289 nm of 1.998 was measured, indicating a concentration of 3.07×10⁻² M (ε=13,000) and 20.5% yield of Compound 24a. A solution of the compound was made with water to a total of 3.07 mL (20 mM) and stored at −4 ° C.

Preparation of Compound 1. Cy5-BOSu₂ (Compound 11, 64 mg, 60 μmol) in 1 M NaHCO₃, pH 8.1 (0.5 mL) was added to a solution of ddUTP(S)-PA—NH₂ (Compound 24a, 10 μmol, 0.5 mL of a 20 mM in 0.5 M Na₂CO₃ solution) in a small reaction vial. The solution was stirred at room temperature for 2 hours and then overnight at 0 to 4° C. The crude reaction was purified using PTLC (silica gel, 2 mm plate, 2:1 CH₂Cl₂:MeOH). The bottom blue band was cut from the plate and the solid was extracted with 2:1 of MeOH:H20 (˜200 mL). The solution was filtered to remove the silica gel solid. The product containing filtrate was then concentrated to dryness and the residue was re-dissolved in water (2 mL). The crude product was subjected to HPLC (C₁₈ column) purification and eluted with gradient solvent of MeOH and 5 mM phosphate, pH 7.0 (15% to 100%, 5 mL/min) and then a DEAE column (30 mL) purification using gradient elution (0.05 M to 1.0 M TEAB buffer, 5 mL/min, total of 1200 mL). The dark blue band was collected in several fractions (10 mL per tube). The purity of the fractions were checked by CE/LIF. The pure fractions were combined and concentrated to dryness. The product was re-dissolved in 1:1 MeOH: H₂O (20 mL) and checked by CE/LIF. The absorbance of the solution (molar absorptivity, ε=203,000), concentration and total quantity of the product was calculated and determined to be at 1.186 μM (23.7% yield of Compound 1).

### Example 3

### Synthetic Preparation of Compound 2

Preparation of ddCTP(S)-propargylamine (24b, ddCTP(S)-PA—NH₂). Referring again to Scheme II, Compound 15b, iodo-dideoxycytidine, obtained by known procedures, was reacted with the propargyl amino compound using palladium coupling chemistry to produce Compound 19b in 94% yield. See, e.g., Bergstrom et al., J. Carbohydrates, Nucleosides and Nucleotides, 4:257 (1977).

Compound 19b (ddC-PA-TFA, 170 mg, 472 μmol) and pyridine (10 mL) were added to an oven dried 25 mL round bottom flask. The reaction was concentrated to dryness under high vacuum and another 10 mL of pyridine was then added and the reaction. The reaction was concentrated to dryness and then dried under high vacuum pump for 2 hours. Triethylphosphate (2 mL) was then added to the flask and the reaction was cooled to 0° C. Thiophosphoryl chloride (PSCl₃, 26, 126 μL, 210.2 mg, 1.24 mmol) and pyridine (90 μL, 92 mg, 1.16 mmol) were then added to the reaction flask and the heterogeneous mixture was stirred at 0 to 4° C. for 5 hours. Bis(tri-n-butylammonium)pyrophosphate (882 mg, 1.9 mmol) in tributylamine (340 μL) and acetonitrile (7.5 mL) were then added to the reaction flask, followed by stirring at room temperature for 30 minutes under nitrogen. The solution was then set aside at room temperature for 24 hours and then concentrated to dryness.

The reaction was purified by co-evaporation of the reaction residue twice with MeOH (10 mL), followed by then dissolving in 0.05 M TEAB buffer, and then purification on a DEAE column (A-25 gel) using gradient (0.1 M to 1 M) TEAB buffer (1200 mL). Each fraction (8 mL) of the solution was collected and checked with CE. The fractions that contained the products (collected after eluting with 0.6 M to 0.7 M of TEAB buffer) were combined and concentrated to dryness. The crude product was then dissolved in water (7 mL) and to it was added NH₄OH (7 mL) to de-protect the TFA group from the amine. The reaction was stirred overnight and the solvent was evaporated to dryness. The residue was re-dissolved in water and analyzed with CE (15 KV, 100 mM borate buffer, pH 10.2). The solution was treated with 3 mL of 1 M Na₂CO₃ and evaporated to dryness to remove residual NH₄OH. The product was then re-dissolved in water (12 mL) and the UV absorbance (1/100 dilution) at λmax 292 nm of 1.0409 was measured, indicating a concentration of 11.19 mM (ε=9,300) in 28.6% yield of Compound 24b.

Preparation of Compound 2. Compound 12 (Cy7-BOSu₂, 80 mg, 81.7 μmol) in 0.1 M NaHCO₃/Na₂CO₃ pH 9.0 buffer (1.5 mL) was added to ddCTP(S)-PA—NH₂ (Compound 24b, 11.19 μmol, 1.0 mL of a 11.19 mM in 0.5 M Na₂CO₃ solution), prepared as described above, in a small vial. Isopropanol (0.75 mL) was then added to the reaction, followed by stirring at room temperature for 4 hours in the dark. The crude reaction product was purified using PTLC (silica gel, three 2 mm plate, 1:1 CHCl₃:MeOH). The bottom blue band was cut from the plate and the solid was extracted with 6:1 of MeOH:H₂O (˜200 mL). The extract was centrifuged and the supernatant solution was filtered through Celite to remove the silica gel solid. The filtrate was concentrated to dryness and the residue was re-dissolved in 10% MeOH/water (2 mL). The crude reaction product was subjected to HPLC (C18 column) purification and eluted with gradient solvent of MeOH and 10 mM phosphate, pH 7.0 (15% to 100%, 5 mL/min), followed by purification on a DEAE column (30 mL) using gradient elution (0.1 M to 1.0 M TEAB buffer, 5 mL/min, total of 1700 mL). The dark blue band was collected in several fractions (10 mL per tube). The purity of each fraction was checked by CE/LIF and the pure fractions were combined and passed through a SPE column to desalt and concentrate the solution. The product was collected by eluting with 1:1 MeOH:H₂O (2 mL) and checking by CE/LIF. The absorbance of the solution was measured (molar absorptivity, ε=170,000), and the concentration and total quantity of the product was calculated and determined to be at 168.24 μM (5 mL, 7.5% yield of Compound 2).

### Example 4

### Synthetic Preparation of Compound 3

Preparation of Compound 19c (ddA-PA-TFA). Referring again to Scheme II, iodo-dideoxy-7-deazaadnosine (Compound 15c) (429.2 mg, 1.19 mmol), obtained by known procedures, CuI (45.4 mg, 0.238 mmol), and DMF (6 mL) were added to an oven dried 100 mL round bottom flask with a stirring bar to form a 0.2 M solution. See, e.g., Moffatt et al, J. Am. Chem. Soc., 95:4016 (1972) and Robins et al., Tetrahedron Lett., 25:367 (1984). The flask was flushed with nitrogen and capped. The propargyl amine side arm (544 mg, 3.6 mmol), triethylamine (0.34 mL, 2.4 mmol) and then Pd(Ph₃P)₄ (138.7 mg, 0.12 mmol) were added to the flask and the reaction was stirred at room temperature under nitrogen atmosphere. The reaction was complete after stirring overnight when monitored with TLC (silica gel plate, 9:1 CH₂Cl₂:MeOH) for the disappearance of starting material (R_{f}=0.45) and the formation of the product (R_{f}=0.36). The reaction was concentrated to dryness, re-dissolved in MeOH (10 mL), and co-evaporated with silica gel (5 g) to dryness. The crude product was then subjected to column chromatography purification (silica gel, eluted with gradient MeOH/CH₂Cl₂ solvent, 0% to 10% MeOH). The product fractions were combined and concentrated to dryness under high vacuum to give 410.5 mg (89.9% yield) of the product, Compound 19c.

Preparation of ddATP(S)-propargylamine (Compound 24c, ddATP(S)-PA—NH₂). ddA-PA-TFA (Compound 19c, 110.7 mg, 289 μmol), prepared as described above, and pyridine (10 mL) were added to an oven dried 25 mL round bottom flask. The reaction was concentrated to dryness under high vacuum and another 10 mL of pyridine was added. The reaction was then concentrated to dryness and dried under high vacuum pump for 2 hours. Pyridine (300 μL) was added to the reaction flask and the flask was briefly flushed with nitrogen via a needle through a rubber septum in the flask. Dioxane (900 μL) was then added to the reaction, followed by 300 μL of a freshly prepared 1 M solution of 2-chloro-4H-1,2,3-dioxaphosphorin-4-one (Compound 7) in anhydrous dioxane. After 30 minutes, 1200 μL of a 0.5 M solution of bis(tri-n-butylammonium)pyrophosphate in 3:1 ratio of DMF and tri-n-butylamine was added and the flask was stirred at room temperature for 25 minutes under nitrogen. A suspension of S₈ (20 mg) in DMF (600 μL) was then added and the reaction was stirred for 30 min. TEAB buffer (1,000 μL of 1 M TEAB) was then added and the reaction mixture was stirred overnight, followed by evaporation of the solvent to dryness under high vacuum.

The crude product was purified with a DEAE column (A-25, 0.05 M TEAB, 2.5 cm×13 cm) using gradient (0.05 M to 1 M) TEAB buffer (1200 mL). Each fraction (8 mL) of the solution was collected and checked with CE. The fractions that contained the product (collected between eluting with 0.66 M to 0.75 M of TEAB buffer) were combined and concentrated to dryness. The crude product was then dissolved in water (6 mL) and NH₄OH (6 mL) was added to de-protect the TFA group from the amine. The reaction was stirred overnight and the solvent was evaporated to dryness. The product was re-dissolved in water and analyzed with CE (15 KV, 100 mM borate buffer, pH 10.2). The solution was treated with 1.5 mL of 1 M Na₂CO₃ and evaporated to dryness to remove residual NH₄OH. The solution was then re-dissolved in water (2 mL) and the UV absorbance (1/800 dilution) at λmax 289 nm of 0.8817 was measured, indicating a concentration of 5.55×10⁻² M (ε=12,700), and 38.4% yield of Compound 24c. A solution of Compound 24c was made with water to a total of 5.55 mL (20 mM) and stored at −4° C.

Preparation of Compound 3. DBCy7-BOSu₂ (Compound 14, 72 mg, 60 μmol) in 1 M NaHCO₃, pH 8.1 (0.5 mL) and ddATP(S)-PA—NH₂ (Compound 24c, 10 μmol, 0.5 mL of a 20 mM in 0.5 M Na₂CO₃ solution), prepared as described above were added to a small vial. The reaction was stirred at room temperature for 2 hours and then overnight at 0 to 4° C. The crude reaction product was purified using PTLC (silica gel, 2 mm plate, 2:1 CH₂Cl₂:MeOH). The bottom blue band was cut off the plate and the solid was extracted with 2:1 of MeOH:H₂O (˜200 mL). The solution was then filtered to remove the silica gel solid and the crude product was checked with CE. The filtrate was concentrated to dryness and the residue was re-dissolved in water (2 mL). The crude product was purified by HPLC (C18 column) and eluted with gradient solvent of MeOH and 5 mM phosphate, pH 7.0 (15% to 100%, 5 mL/min), followed by a DEAE column (30 mL) purification using gradient elution (0.05 M to 1.0 M TEAB buffer, 5 mL/min, total of 1200 mL). The dark blue-green band was collected in several fractions (10 mL per tube) and the purity of each fraction was checked by CE/LIF. The pure fractions were combined and concentrated to dryness. The product was re-dissolved in 1:1 MeOH:H₂O (20 mL) and the absorbance of the solution (molar absorptivity ε=171,000) was measured. The concentration and total quantity of the product was calculated and determined. The product was concentrated and re-dissolved in a 1:1 MeOH:H₂O solution to make a stock solution with concentration of 200 μM.

### Example 5

### Synthetic Preparation of Compound 4

Preparation of ddGTP(S)-propargylamine (Compound 24d, ddGTP(S)-PA—NH₂). Referring again to Scheme II, Compound 15d, iodo-dideoxy-7-deazaguanosine, obtained by known procedures, was reacted with the propargyl amino compound using palladium coupling chemistry to produce Compound 19d in 94% yield. See, e.g., Hobbs et al., U.S. Pat. No. 5,151,507 and references cited therein.

ddG-PA-TFA (Compound 19d, 150 mg, 0.376 mmol) and pyridine (10 mL) were added to an oven dried 25 mL round bottom flask and the reaction was concentrated to dryness under high vacuum. Another 10 mL of pyridine was added and the reaction was again concentrated to dryness. The flask was then dried under high vacuum pump for 2 hours. Triethylphosphate (2 mL) was added and the reaction was cooled to 0° C. Thiophosphoryl chloride (PSCl₃, 26, 100 μL, 166.8 mg, 0.98 mmol) and pyridine (70 μL, 68.5 mg, 0.87 mmol) were then added. The heterogeneous mixture was stirred at 0 to 4° C. for 5 hours followed by addition of a solution of bis(tri-n-butylammonium)pyrophosphate (700 mg, 1.5 mmol) in tributylamine (270 μL) and acetonitrile (6 mL). The flask was stirred at room temperature for 30 minutes under nitrogen. The solution was then set aside at room temperature for 24 hours and then concentrated to dryness.

The residue was co-evaporated twice with MeOH (10 mL) and then dissolved in 0.05 M TEAB buffer and then subjected to a DEAE column (A-25 gel) separation using gradient (0.1 M to 1 M) TEAB buffer (1200 mL). Each fraction (8 mL) of the solution was collected and checked with CE. The fractions that contained the product (collected after eluting with 0.6 M to 0.7 M of TEAB buffer) were combined and concentrated to dryness. The crude product was then dissolved in water (10 mL) and to it was added NH₄OH (10 mL) to de-protect the TFA group from the amine. The reaction was stirred overnight and the solvent was evaporated to dryness. The product was re-dissolved in water and analyzed with CE (15 KV, 100 mM borate buffer, pH 10.2). The solution was treated with 10 mL of 1 M Na₂CO₃ and evaporated to dryness to remove residual NH₄OH. The product was then re-dissolved in water (10 mL) and the UV absorbance (1/200 dilution) at λmax 292 nm of 0.576 was measured, indicating a concentration of 9.58 mM (ε=11,900) and a 25.5% yield of Compound 19d.

Preparation of Compound 4. ddGTP(S)-PA—NH₂ (Compound 19d, 12 μmol, 1.25 mL of a 9.58 mM in 1.0 M Na₂CO₃ solution), prepared as described above, was added to a solution of DBCy5-BOSu₂ (Compound 13, 76 mg, 72 μmol) in 0.1 M NaHCO₃/Na₂CO₃ pH 9.0 buffer (1.5 mL) in a small vial, followed by the addition of isopropanol (0.6 mL). The reaction was stirred at room temperature for 4 hours in the dark. The crude reaction product was purified using PTLC (silica gel, three 2 mm plate, 1:1 CHCl₃:MeOH). The bottom blue band was cut off the plate and the solid was extracted with 6:1 of MeOH:H₂O (˜200 mL). The solid was centrifuged and the supernatant was filtered through Celite to remove the silica gel solid. The filtrate was concentrated to dryness and the residue was re-dissolved in 0.1 M TEAB buffer (3 mL). The crude product was checked with CE and then purified on a DEAE column (30 mL) using gradient elution (0.1 M to 1.0 M TEAB buffer, 5 mL/min, total of 1700 mL). The dark blue band was collected in several fractions (10 mL per tube). The purity of each fraction was checked by CE/LIF. The pure fractions were combined and passed through a SPE column to desalt and concentrate the solution. The product was collected by eluting with 1:1 MeOH:H₂O (2 mL) and checked by CE/LIF. The absorbance of the solution (molar absorptivity ε=224,000) was measured and the concentration and total quantity of the product was calculated and determined to be at 1173.64 μM (12 mL, 17.4% yield of Compound 4).

### Example 6

### Spectral Measurements

The absorbance and emission spectra of Compounds 1-4 was measured using a Beckman DU7400 spectrophotometer in water media. The results are summarized in Table 1.

TABLE 1
Absorbance of Compounds 1-4 and Corresponding Cyanine Dyes.

| | | Cyanine | | |
|---|---|---|---|---|
| Dye-Terminators | Abs (H₂O) | Dyes | Abs (H₂O) | Em (H₂O) |
| Cy5-ddUTP(S) (1) | 651 nm | Cy5 | 649 nm | 675 nm |
| DBCy5-ddGTP(S) (4) | 687 nm | DBCy5 | 682 nm | 708 nm |
| Cy7-ddCTP(S) (2) | 753 nm | Cy7 | 750 nm | 785 nm |
| DBCy7-ddATP(S) (3) | 787 nm | DBCy7 | 784 nm | 814 nm |

As shown in Table 1 above, the maximum absorbance for each of the four new [symbol] -(thio)triphosphate dye terminators (Compounds 14) is about the same as that of the corresponding free cyanine dye. Thus, using existing automated sequencer systems, such as the Beckman CEQ™ automated sequencers, to evaluate these dye terminators does not require any additional modification of the hardware on these systems. As shown in Table 1 above, the absorbance maxima of the peaks are grouped into two pairs. Cy5 and DBCy5 are one pair and separated by 33 nm. Cy7 and DBCy7 are another pair and separated by 34 nm in wavelength. The two pairs of dyes are separated by approximately 100 nm apart. Accordingly, the use of two diode laser sources to excite the two pairs of dyes is feasible as is used in currently available sequencing instruments.

Although the present invention has been discussed in considerable detail with reference to certain preferred embodiments, other embodiments are possible. Therefore, the scope of the appended claims should not be limited to the description of preferred embodiments contained herein.

What is claimed is:

1. A compound of the formula: 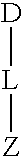
wherein
Z is a (thio)triphosphate nucleotide derivative;
D is a fluorescent dye; and
L is a linker of sufficient length to connect the (thio)triphosphate nucleotide to the fluorescent dye, such that the fluorescent dye does not significantly interfere with the overall binding and recognition of the nucleotide derivative by a nucleic acid replication enzyme.

2. A compound according to claim 1 wherein Z is an α-(thio)triphosphate dideoxynucleotide.

3. A compound according to claim 1 wherein D is a dye having a near infrared fluorescence.

4. A compound according to claim 3 wherein D is a fluorescent cyanine dye.

5. A compound according to claim 4 wherein D is a fluorescent cyanine dye of the formula: 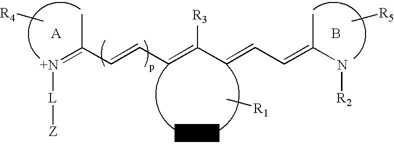
wherein
Z is defined as in claim 1;
A and B are each independently ring structures having sufficient atoms to form a cyanine nuclei;
n is 0, 2, 3, or 4;
p is 0 or 1;
R₁ is selected from the group consisting of C₁-C₄ alkyl, C₁-C₄ haloalkyl, halogen, and hydroxy with the proviso that R₁ is not present when n is 0;
R₂ is selected from the group consisting of C₁-C₂₀ alkyl and hydrogen, wherein the C₁-C₂₀ alkyl is unsubstituted or substituted with C₁-C₄ alkoxy, C₁-C₄ alkoxycarbonyl, C₁-C₄ alkyl, amine, amide, C₁-C₄ carboxy, C₁-C₄ carboxylate, halogen, hydroxy, and sulfonate;
R₃ is selected from the group consisting of hydrogen, halogen, OR₇, SR₇, NR₇R₈, C₁-C₆ alkyl, C₁-C₆ alkylene, C₃-C₆ cycloalkyl, C₃-C₆ cycloheteroalkyl, C₃-C₆ cycloalkylene, C₃-C₆ cycloheteroalkylene, phenyl, biaryl, heteroaryl, or heterobiaryl, wherein the C₁-C₆ alkyl, C₁-C₆ alkylene, C₃-C₆ cycloalkyl, C₃-C₆ cycloheteroalkyl, C₃-C₆ cycloalkylene, C₃-C₆ cycloheteroalkylene, phenyl, biaryl, heteroaryl and heterobiaryl groups are unsubstituted or substituted with C₁-C₄ alkyl, C₁-C₄ haloalkyl, halogen or hydroxy;
R₄ and R₅ are each independently selected from the group consisting of C₁-C₄ alkoxy, C₁-C₄ alkoxycarbonyl, C₁-C₄ alkyl, amine, amide, C₁-C₄ carboxy, C₁-C₄ carboxylate, halogen, hydroxy, and sulfonate;
R₇ and R₈ are each independently selected from the group consisting of hydrogen, C₁-C₆ alkyl, C₃-C₆ cycloalkyl, phenyl, biaryl, heteroaryl, and heterobiaryl, wherein the C₁-C₆ alkyl, C₁-C₆ cycloalkyl, phenyl, biaryl, heteroaryl, and heterobiaryl groups may be substituted with halogen, OH, C₁-C₄ alkyl, and C₁-C₄ haloalkyl, or when R₃ represents NR₇R₈, R₇ and R₈ may be taken together to form an optionally substituted
C₃-C₆ aliphatic or C₃-C₆ aromatic heterocyclic ring.

6. A compound according to claim 1 wherein Z is selected from the group consisting of:

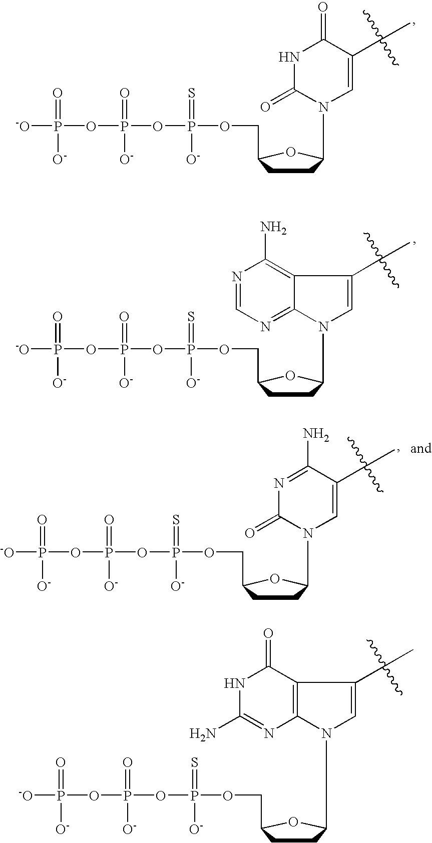

7. A compound according to claim 1 wherein L is a linker of the formula: 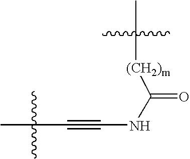
wherein m is an integer from 1 to 20.

8. A compound according to claim 1 of the formula: 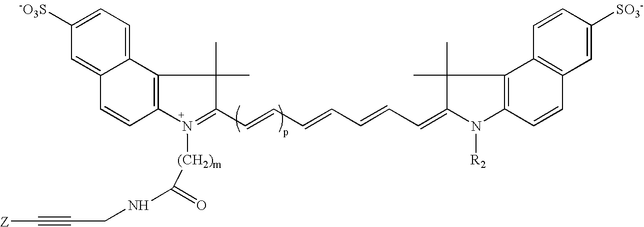
wherein
Z is defined as in claim 1;
m is an integer from 1 to 20.
p is 0 or 1;and
R₂ is selected from the group consisting of C₁-C₂₀ alkyl and hydrogen, wherein the C₁-C₂₀ alkyl is unsubstituted or substituted with C₁-C₄ alkoxy, C₁-C₄ alkoxycarbonyl, C₁-C₄ alkyl, amine, amide, C₁-C₄ carboxy, C₁-C₄ carboxylate, halogen, hydroxy, and sulfonate.

9. A compound according to claim 1 of the formula: 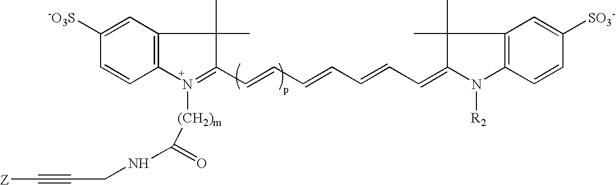
wherein
Z is defined as in claim 1;
m is an integer from 1 to 20;
p is 0 or 1;and
R₂ is selected from the group consisting of C₁-C₂₀ alkyl and hydrogen, wherein the C₁-C₂₀ alkyl is unsubstituted or substituted with C₁-C₄ alkoxy, C₁-C₄ alkoxycarbonyl, C₁-C₄ alkyl, amine, amide, C₁-C₄ carboxy, C₁-C₄ carboxylate, halogen, hydroxy, and sulfonate.

10. A compound according to claim 1 of the formula: 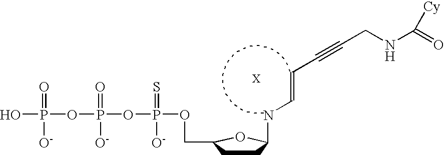
wherein
X are the atoms sufficient to make up a heterocyclic base; and
Cy is a fluorescent dye.

11. A compound according to claim 10 wherein Cy is a cyanine dye.

12. A method of nucleic acid sequence analysis comprising:
reacting a compound according to claim 1 with a first nucleic acid sequence to produce a second nucleic acid sequence labeled with the fluorescent reporter-labeled compound; and
detecting the reporter on the second nucleic acid sequence.

13. A method for determining the base sequence of a target DNA comprising:
providing a mixture of compounds according to claim 1, the mixture of compounds corresponding to each of the four DNA bases;
providing a DNA template corresponding to the target DNA;
reacting the DNA template with a DNA primer;
extending the DNA template and DNA primer with a replication enzyme, a mixture of DNA nucleotides, and the mixture of compounds to produce a plurality of DNA fragments, each DNA fragment having a fluorescent reporter labeled compound attached to the 3′-terminal residue of the DNA fragment;
separating the plurality of fluorescent reporter labeled DNA fragments; and
detecting the fluorescent reporter on each separated fluorescent reporter labeled DNA fragment to determine the base sequence of the target DNA.

14. A method of nucleic acid sequence analysis comprising:
providing a template nucleic acid;
providing one or more fluorescent reporter labeled (thio)triphosphate nucleotide compounds;
reacting a nucleotide primer with a replication enzyme, a mixture of nucleotide precursors, and the one or more fluorescent reporter labeled (thio)triphosphate nucleotide compounds to extend the nucleotide primer and produce a plurality of nucleotide fragments having a fluorescent reporter labeled (thio)triphosphate nucleotide compound attached to the 3′-terminal residue of each nucleotide fragment;
separating the plurality of nucleotide fragments; and
detecting the fluorescent reporter for each separated fluorescent reporter labeled nucleotide fragment.

15. A method according to claim 14 wherein at least one of the fluorescent reporter labeled (thio)triphosphate nucleotide compounds is labeled with a cyanine dye.

16. A method according to claim 14 wherein at least one of the fluorescent reporter labeled (thio)triphosphate nucleotide compounds is labeled with a dye having a near infrared fluorescence.

17. A method according to claim 14 wherein four different fluorescent reporter labeled (thio)triphosphate nucleotide compounds are provided, each fluorescent reporter labeled chain terminating (thio)triphosphate nucleotide compound being labeled with a dye having a near infrared fluorescence.

18. A method according to claim 17 wherein each of the four different fluorescent reporter labeled (thio)triphosphate nucleotide compounds is labeled with a cyanine dye.
